(19) United States
(12) Patent Application Publication
(10) Pub. No.: US 2024/0398246 A1
(43) Pub. Date: Dec. 5, 2024

(54) GASBAG DETECTION METHOD AND APPARATUS FOR WEARABLE DEVICE, AND WEARABLE DEVICE

(71) Applicant: Huawei Technologies Co., Ltd., Shenzhen (CN)

(72) Inventor: Hao Yuan, Xi’an (CN)

(21) Appl. No.: 18/699,536

(22) PCT Filed: Aug. 23, 2022
(86) PCT No.: PCT/CN2022/114113

§ 371 Date: Apr. 8, 2024

(30) Foreign Application Priority Data
Oct. 9, 2021 (CN) 202111176128.X

(51) Int. Cl. A61B 5/022; A61B 5/00

(52) U.S. Cl. CPC A61B 5/02241; A61B 5/681; A61B 5/6844; A61B 2562/0247

(57) ABSTRACT

A gasbag detection method and apparatus for a wearable device, and the wearable device are provided. The method includes: when a gasbag is mounted on the wearable device, detecting, based on a wrist circumference of a user, whether the gasbag mounted on the wearable device matches the wrist circumference of the user; and if the gasbag mounted on the wearable device does not match the wrist circumference of the user, outputting first prompt information, where the first prompt information indicates to mount a gasbag that matches the wrist circumference of the user.

### CROSS-REFERENCE TO RELATED APPLICATIONS

This application is a national stage of International Application No. PCT/CN2022/114113, filed on Aug. 23, 2022, which claims priority to Chinese Patent Application No. 202111176128.X, filed on Oct. 9, 2021. Both of the aforementioned applications are hereby incorporated by reference in their entireties.

### TECHNICAL FIELD

Embodiments of this application relate to wearable device technologies, and in particular, to a gasbag detection method and apparatus for a wearable device, and the wearable device.

### BACKGROUND

With development of wearable devices, a current wearable device may provide a user with abundant function services. For example, if the wearable device is a watch, the watch may not only display time, but also provide the user with function services such as communication, entertainment, blood pressure measurement, and heart rate measurement.

When the user measures a blood pressure by using the watch, a gasbag disposed on the watch may be inflated by using the watch. If the inflated gasbag is small and cannot fully cover a wrist of the user, a blood pressure measurement result is inaccurate.

### SUMMARY

Embodiments of this application provide a gasbag detection method and apparatus for a wearable device, and the wearable device, to improve accuracy of blood pressure detection.

According to a first aspect, an embodiment of this application provides a gasbag detection method for a wearable device. An execution body of the method is the wearable device or a chip in the wearable device. The following uses an example in which the execution body of the method is the wearable device for description. In an embodiment, the wearable device may include but is not limited to a device that is used to be worn on a wrist of a user, like a watch or a band.

In the method, when a gasbag is mounted on the wearable device, the wearable device may detect, based on a wrist circumference of the user, whether the gasbag mounted on the wearable device matches the wrist circumference of the user; and if the gasbag mounted on the wearable device does not match the wrist circumference of the user, output first prompt information, where the first prompt information indicates to mount a gasbag that matches the wrist circumference of the user. After the user mounts, on the wearable device, the gasbag that matches the wrist circumference of the user, the user can accurately measure a blood pressure. In an embodiment, the first prompt information includes an identifier of the gasbag that matches the wrist circumference of the user.

If the gasbag mounted on the wearable device matches the wrist circumference of the user, the wearable device may inflate the gasbag by using an air pump in the wearable device, to detect the blood pressure of the user.

In an embodiment, when the user needs to measure the blood pressure, the user may input the wrist circumference and an instruction for measuring the blood pressure in the wearable device. In an embodiment, when the user needs to measure blood pressure, if the wrist circumference is output, the user may input an instruction for measuring the blood pressure. In response to receiving an instruction indicating that the user inputs the wrist circumference, or receiving the instruction that is input by the user and that is for measuring the blood pressure, the wearable device may detect, based on the wrist circumference of the user, whether the gasbag mounted on the wearable device matches the wrist circumference of the user.

It should be understood that wrists of different users have different thicknesses, namely, different wrist circumference. For a user with a large wrist circumference, if a small-sized gasbag is used, the gasbag cannot fully cover a wrist of the user, and a blood pressure measurement result is inaccurate. For a user with a small wrist circumference, a large-sized gasbag can fully cover the wrist of the user, and a blood pressure measurement result is accurate. However, for the user with the small wrist circumference, a small-sized gasbag can fully cover the wrist of the user, and the small-sized gasbag is more convenient for use. Therefore, in this embodiment of this application, when the user performs blood pressure measurement by using the wearable device, it is important to recommend an appropriate gasbag to the user. This can improve user experience.

A premise of that the wearable device may detect, based on the wrist circumference of the user, whether the gasbag mounted on the wearable device matches the wrist circumference of the user is that there is a gasbag mounted on the wearable device. Therefore, the wearable device further needs to detect whether the gasbag is mounted on the wearable device. Further, in response to detecting that the gasbag is mounted on the wearable device, the wearable device detects an identifier of the gasbag mounted on the wearable device, and detects, based on a mapping relationship between a wrist circumference and an identifier of a gasbag, whether the identifier of the gasbag mounted on the wearable device is the identifier of the gasbag mapped to the wrist circumference of the user. It should be understood that the wearable device pre-stores the mapping relationship between the wrist circumference and the identifier of the gasbag.

If the identifier of the gasbag mounted on the wearable device is not the identifier of the gasbag mapped to the wrist circumference of the user, the wearable device determines that the gasbag mounted on the wearable device does not match the wrist circumference of the user; or if the identifier of the gasbag mounted on the wearable device is the identifier of the gasbag mapped to the wrist circumference of the user, the wearable device determines that the gasbag mounted on the wearable device matches the wrist circumference of the user.

In an embodiment, the identifier of the gasbag may be, but is not limited to, information for distinguishing the gasbag, such as a type, a volume, and a number of the gasbag.

The following describes, from three aspects, a process in which the wearable device detects whether the gasbag is mounted on the wearable device, and detects the identifier of the gasbag mounted on the wearable device.

In a second manner, the wearable device includes a Hall effect sensor, and the Hall effect sensor includes a positive pin, a negative pin, and a signal output pin. In response to sensing a positive magnetic pole, the positive pin outputs, through the signal output pin, a level greater than a second threshold; in response to sensing a negative magnetic pole, the negative pin outputs, through the signal output pin, the level greater than the second threshold; when the positive pin does not sense the positive magnetic pole, the positive pin outputs, through the signal output pin, a level less than a first threshold, where the second threshold is greater than the first threshold; and when the negative pin does not sense the negative magnetic pole, the negative pin outputs, through the signal output pin, the level less than the first threshold.

In other words, the Hall effect sensor may be configured to detect the positive magnetic pole or the negative magnetic pole.

In this manner, the wearable device includes at least two gasbags, magnetic poles are disposed on the gasbags of the at least two gasbags, and a different magnetic pole is disposed on each gasbag.

The wearable device may detect, based on the level output through the signal output pin, whether the gasbag is mounted on the wearable device. The wearable device may detect, based on the pin that outputs, through the signal output pin, the level greater than the second threshold, the identifier of the gasbag mounted on the wearable device.

For example, the at least two gasbags include a first gasbag and a second gasbag, the negative magnetic pole is disposed on the first gasbag, and the positive magnetic pole is disposed on the second gasbag. In this example, if the level output through the signal output pin is less than the first threshold, the wearable device determines that no gasbag is mounted on the wearable device. If the level output through the signal output pin is greater than the second threshold, the wearable device determines that the gasbag is mounted on the wearable device.

If the positive pin outputs, through the signal output pin, the level greater than the second threshold, the wearable device determines that the gasbag mounted on the wearable device is the second gasbag. If the negative pin outputs, through the signal output pin, the level greater than the second threshold, the wearable device determines that the gasbag mounted on the wearable device is the first gasbag.

In a second manner, the wearable device includes a differential pressure sensor and a gasbag watchband, the differential pressure sensor is configured to detect a differential pressure at an air nozzle of an air pump in the wearable device, the gasbag is mounted on the gasbag watchband, and when the gasbag watchband on which the gasbag is mounted is mounted on the wearable device, an air intake on the gasbag communicates with the air nozzle. Because volumes of gasbags with different identifiers are different, when the wearable device performs inflation by using the air pump, a change rate of the differential pressure of the gasbag is different. In this embodiment of this application, a change rate of the differential pressure at the air nozzle is used to represent the change rate of the differential pressure of the gasbag.

Specifically, the wearable device may control the air pump to blow air at a preset rate; obtain a change rate of the differential pressure within second preset duration based on the differential pressure at the air nozzle collected by the differential pressure sensor; further detect, based on the change rate of the differential pressure within the second preset duration, whether the gasbag is mounted on the wearable device; and detect, based on the change rate of the differential pressure within the second preset duration, the identifier of the gasbag mounted on the wearable device.

It should be understood that the wearable device further includes a non-gasbag watchband, and when the non-gasbag watchband is mounted on the wearable device, the non-gasbag watchband blocks the air nozzle of the air pump. Therefore, in the foregoing case, if the change rate of the differential pressure within the second preset duration is o, or the change rate of the differential pressure within the second preset duration is greater than or equal to a first change rate threshold, the wearable device determines that no gasbag is mounted on the wearable device (in this case, no watchband is mounted on the wearable device, or the non-gasbag watchband is mounted on the wearable device).

If the change rate of the differential pressure within the second preset duration is less than the first change rate threshold, the wearable device determines that the gasbag is mounted on the wearable device. Further, in response to information that the change rate of the differential pressure within the second preset duration is less than the first change rate threshold, the wearable device detects, based on the change rate of the differential pressure within the second preset duration and a mapping relationship between a change rate of a differential pressure and an identifier of a gasbag, the identifier of the gasbag mounted on the wearable device.

In a possible implementation, the differential pressure sensor includes a first pressure sensor and a second pressure sensor, the first pressure sensor is configured to detect a first pressure at the air nozzle, the second pressure sensor is configured to detect a second pressure, the second pressure is an environmental pressure, and the differential pressure at the air nozzle is a difference between the first pressure and the second pressure.

In this embodiment of this application, the wearable device may obtain once the first pressure collected by the first pressure sensor at an interval of first preset duration, and obtain once the second pressure collected by the second pressure sensor at the interval of first preset duration. The wearable device obtains a first pressure average value based on first pressures collected within the second preset duration, obtains a second pressure average value based on second pressures collected within the second preset duration, and further obtains the change rate of the differential pressure within the second preset duration based on the first pressure average value and the second pressure average value.

In a third manner, the wearable device includes: the Hall effect sensor in the first manner, the differential pressure sensor, the gasbag watchband, and the at least two gasbags, the differential pressure sensor is configured to detect the differential pressure at the air nozzle of the air pump in the wearable device, the gasbag is mounted on the gasbag watchband, when the gasbag watchband on which the gasbag is mounted is mounted on the wearable device, the air intake on the gasbag communicates with the air nozzle, and magnetic poles disposed on at least two of the following are the same: the first gasbag in the at least two gasbags, the second gasbag in the at least two gasbags, and the non-gasbag watchband.

In this manner, the wearable device may detect, with reference to the Hall effect sensor and the change rate of the differential pressure of the gasbag, whether the gasbag is mounted on the wearable device, and detect the identifier of the gasbag mounted on the wearable device.

Specifically, the wearable device may control the air pump to blow air at the preset rate; obtain the change rate of the differential pressure within the second preset duration based on the differential pressure at the air nozzle collected by the differential pressure sensor; and detect, based on the level output through the signal output pin and the change rate of the differential pressure within the second preset duration, whether the gasbag is mounted on the wearable device; and the wearable device may detect, based on the pin that outputs, through the signal output pin, the level greater than the second threshold and the change rate of the differential pressure within the second preset duration, the identifier of the gasbag mounted on the wearable device.

For example, the negative magnetic pole is disposed on the first gasbag, the positive magnetic pole is disposed on the second gasbag, and the positive magnetic pole is disposed on the non-gasbag watchband. If the level output through the signal output pin is less than the first threshold, the wearable device determines that no gasbag is mounted on the wearable device. If the level output through the signal output pin is greater than the second threshold, and the negative pin outputs, through the signal output pin, the level greater than the second threshold, the wearable device determines that the gasbag mounted on the wearable device is the first gasbag. If the level output through the signal output pin is greater than the second threshold, and the positive pin outputs, through the signal output pin, the level greater than the second threshold, the wearable device detects, based on the change rate of the differential pressure within the second preset duration, that the second gasbag or the non-gasbag watchband is mounted on the wearable device.

When the non-gasbag watchband is mounted on the wearable device, the non-gasbag watchband blocks the air nozzle of the air pump. In this manner, if the change rate of the differential pressure within the second preset duration is greater than or equal to the first change rate threshold, the wearable device determines that the non-gasbag watchband is mounted on the wearable device. If the change rate of the differential pressure within the second preset duration is less than the first change rate threshold, the wearable device determines that the gasbag mounted on the wearable device is the second gasbag.

According to a second aspect, an embodiment of this application provides a gasbag detection apparatus for a wearable device. The gasbag detection apparatus for the wearable device may be a wearable device, or a chip or a processor in the wearable device. The gasbag detection apparatus for the wearable device includes:

a detection module, configured to: when a gasbag is mounted on the wearable device, detect, based on a wrist circumference of a user, whether the gasbag mounted on the wearable device matches the wrist circumference of the user; and

an output module, configured to: if the gasbag mounted on the wearable device does not match the wrist circumference of the user, output first prompt information, where the first prompt information indicates to mount a gasbag that matches the wrist circumference of the user.

In a possible implementation, the detection module is further configured to detect whether the gasbag is mounted on the wearable device.

The detection module is specifically configured to: in response to detecting that the gasbag is mounted on the wearable device, detect an identifier of the gasbag mounted on the wearable device, and detect, based on a mapping relationship between a wrist circumference and an identifier of a gasbag, whether the identifier of the gasbag mounted on the wearable device is an identifier of a gasbag mapped to the wrist circumference of the user.

In a possible implementation, the detection module is specifically configured to: if the identifier of the gasbag mounted on the wearable device is not the identifier of the gasbag mapped to the wrist circumference of the user, determine that the gasbag mounted on the wearable device does not match the wrist circumference of the user; or if the identifier of the gasbag mounted on the wearable device is the identifier of the gasbag mapped to the wrist circumference of the user, determine that the gasbag mounted on the wearable device matches the wrist circumference of the user.

In a possible implementation, the wearable device includes a Hall effect sensor, and the Hall effect sensor includes a positive pin, a negative pin, and a signal output pin.

In response to sensing a positive magnetic pole, the positive pin outputs, through the signal output pin, a level greater than a second threshold; in response to sensing a negative magnetic pole, the negative pin outputs, through the signal output pin, the level greater than the second threshold; when the positive pin does not sense the positive magnetic pole, the positive pin outputs, through the signal output pin, a level less than a first threshold, where the second threshold is greater than the first threshold; and when the negative pin does not sense the negative magnetic pole, the negative pin outputs, through the signal output pin, the level less than the first threshold.

In a possible implementation, the wearable device includes at least two gasbags, magnetic poles are disposed on the gasbags of the at least two gasbags, and a different magnetic pole is disposed on each gasbag.

The detection module is specifically configured to: detect, based on the level output through the signal output pin, whether the gasbag is mounted on the wearable device; and detect, based on the pin that outputs, through the signal output pin, the level greater than the second threshold, the identifier of the gasbag mounted on the wearable device.

In a possible implementation, the at least two gasbags include a first gasbag and a second gasbag, the negative magnetic pole is disposed on the first gasbag, and the positive magnetic pole is disposed on the second gasbag.

The detection module is specifically configured to: if the level output through the signal output pin is less than the first threshold, determine that no gasbag is mounted on the wearable device; or if the level output through the signal output pin is greater than the second threshold, determine that the gasbag is mounted on the wearable device.

In a possible implementation, if the positive pin outputs, through the signal output pin, the level greater than the second threshold, it is determined that the gasbag mounted on the wearable device is the second gasbag; or if the negative pin outputs, through the signal output pin, the level greater than the second threshold, the detection module is specifically configured to determine that the gasbag mounted on the wearable device is the first gasbag.

In a possible implementation, the wearable device includes a differential pressure sensor and a gasbag watchband, the differential pressure sensor is configured to detect a differential pressure at an air nozzle of an air pump in the wearable device, the gasbag is mounted on the gasbag watchband, and when the gasbag watchband on which the gasbag is mounted is mounted on the wearable device, an air intake on the gasbag communicates with the air nozzle.

The detection module is specifically configured to: control the air pump to blow air at a preset rate; obtain a change rate of the differential pressure within second preset duration based on the differential pressure at the air nozzle collected by the differential pressure sensor; detect, based on the change rate of the differential pressure within the second preset duration, whether the gasbag is mounted on the wearable device; and detect, based on the change rate of the differential pressure within the second preset duration, the identifier of the gasbag mounted on the wearable device.

In a possible implementation, the wearable device further includes a non-gasbag watchband, and when the non-gasbag watchband is mounted on the wearable device, the non-gasbag watchband blocks the air nozzle of the air pump.

If the change rate of the differential pressure within the second preset duration is o, or the change rate of the differential pressure within the second preset duration is greater than or equal to a first change rate threshold, the detection module is specifically configured to determine that no gasbag is mounted on the wearable device; or if the change rate of the differential pressure within the second preset duration is less than the first change rate threshold, the detection module is specifically configured to determine that the gasbag is mounted on the wearable device.

In a possible implementation, the detection module is specifically configured to: in response to information that the change rate of the differential pressure within the second preset duration is less than the first change rate threshold, detect, based on the change rate of the differential pressure within the second preset duration and a mapping relationship between a change rate of a differential pressure and an identifier of a gasbag, the identifier of the gasbag mounted on the wearable device.

In a possible implementation, the wearable device includes: a differential pressure sensor, a gasbag watchband, and at least two gasbags, the differential pressure sensor is configured to detect a differential pressure at an air nozzle of an air pump in the wearable device, the gasbag is mounted on the gasbag watchband, when the gasbag watchband on which the gasbag is mounted is mounted on the wearable device, an air intake on the gasbag communicates with the air nozzle, and magnetic poles disposed on at least two of the following are the same: a first gasbag in the at least two gasbags, a second gasbag in the at least two gasbags, and a non-gasbag watchband.

The detection module is specifically configured to: control the air pump to blow air at a preset rate; obtain a change rate of the differential pressure within second preset duration based on the differential pressure at the air nozzle collected by the differential pressure sensor; detect, based on the level output through the signal output pin and the change rate of the differential pressure within the second preset duration, whether the gasbag is mounted on the wearable device; and detect, based on the pin that outputs, through the signal output pin, the level greater than the second threshold and the change rate of the differential pressure within the second preset duration, the identifier of the gasbag mounted on the wearable device.

In a possible implementation, the negative magnetic pole is disposed on the first gasbag, the positive magnetic pole is disposed on the second gasbag, and the positive magnetic pole is disposed on the non-gasbag watchband.

The detection module is specifically configured to: if the level output through the signal output pin is less than the first threshold, determine that no gasbag is mounted on the wearable device; if the level output through the signal output pin is greater than the second threshold, and the negative pin outputs, through the signal output pin, the level greater than the second threshold, determine that the gasbag mounted on the wearable device is the first gasbag; or if the level output through the signal output pin is greater than the second threshold, and the positive pin outputs, through the signal output pin, the level greater than the second threshold, detect, based on the change rate of the differential pressure within the second preset duration, that the second gasbag or the non-gasbag watchband is mounted on the wearable device.

In a possible implementation, when the non-gasbag watchband is mounted on the wearable device, the non-gasbag watchband blocks the air nozzle of the air pump.

The detection module is specifically configured to: if the change rate of the differential pressure within the second preset duration is greater than or equal to the first change rate threshold, determine that the non-gasbag watchband is mounted on the wearable device; or if the change rate of the differential pressure within the second preset duration is less than the first change rate threshold, determine that the gasbag mounted on the wearable device is the second gasbag.

In a possible implementation, the differential pressure sensor includes a first pressure sensor and a second pressure sensor, the first pressure sensor is configured to detect a first pressure at the air nozzle, the second pressure sensor is configured to detect a second pressure, the second pressure is an environmental pressure, and the differential pressure at the air nozzle is a difference between the first pressure and the second pressure.

The detection module is specifically configured to: obtain once the first pressure collected by the first pressure sensor at an interval of first preset duration; obtain once the second pressure collected by the second pressure sensor at the interval of first preset duration; obtain a first pressure average value based on first pressures collected within the second preset duration; obtain a second pressure average value based on second pressures collected within the second preset duration; and obtain the change rate of the differential pressure within the second preset duration based on the first pressure average value and the second pressure average value.

In a possible implementation, a sending and receiving module is configured to: receive an instruction indicating that the user inputs the wrist circumference, or receive an instruction that is input by the user and that is for measuring a blood pressure.

According to a third aspect, an embodiment of this application provides a wearable device, including a Hall effect sensor. The Hall effect sensor includes a positive pin, a negative pin, and a signal output pin.

In response to sensing a positive magnetic pole, the positive pin outputs, through the signal output pin, a level greater than a second threshold; in response to sensing a negative magnetic pole, the negative pin outputs, through the signal output pin, the level greater than the second threshold; when the positive pin does not sense the positive magnetic pole, the positive pin outputs, through the signal output pin, a level less than a first threshold, where the second threshold is greater than the first threshold; and when the negative pin does not sense the negative magnetic pole, the negative pin outputs, through the signal output pin, the level less than the first threshold.

In a possible implementation, the wearable device includes at least two gasbags and a microprocessor unit MCU, and the MCU is connected to the Hall effect sensor; and the MCU is configured to detect the level output through the signal output pin.

In a possible implementation, magnetic poles are disposed on the gasbags of the at least two gasbags, and a different magnetic pole is disposed on each gasbag.

The MCU is further configured to: detect, based on the level output through the signal output pin, whether the gasbag is mounted on the wearable device; and detect, based on the pin that outputs, through the signal output pin, the level greater than the second threshold, an identifier of the gasbag mounted on the wearable device.

In a possible implementation, the wearable device further includes a differential pressure sensor and a gasbag watchband, the differential pressure sensor is configured to detect a differential pressure at an air nozzle of an air pump in the wearable device, the gasbag is mounted on the gasbag watchband, and when the gasbag watchband on which the gasbag is mounted is mounted on the wearable device, an air intake on the gasbag communicates with the air nozzle.

The MCU is further configured to: control the air pump to blow air at a preset rate; obtain a change rate of the differential pressure within second preset duration based on the differential pressure at the air nozzle collected by the differential pressure sensor; detect, based on the change rate of the differential pressure within the second preset duration, whether the gasbag is mounted on the wearable device; and detect, based on the change rate of the differential pressure within the second preset duration, the identifier of the gasbag mounted on the wearable device.

In a possible implementation, the wearable device further includes: a differential pressure sensor, a gasbag watchband, and the at least two gasbags, the differential pressure sensor is configured to detect a differential pressure at an air nozzle of an air pump in the wearable device, the gasbag is mounted on the gasbag watchband, when the gasbag watchband on which the gasbag is mounted is mounted on the wearable device, an air intake on the gasbag communicates with the air nozzle, and magnetic poles disposed on at least two of the following are the same: a first gasbag in the at least two gasbags, a second gasbag in the at least two gasbags, and a non-gasbag watchband.

The MCU is further configured to: control the air pump to blow air at a preset rate; obtain a change rate of the differential pressure within second preset duration based on the differential pressure at the air nozzle collected by the differential pressure sensor; detect, based on the level output through the signal output pin and the change rate of the differential pressure within the second preset duration, whether the gasbag is mounted on the wearable device; and detect, based on the pin that outputs, through the signal output pin, the level greater than the second threshold and the change rate of the differential pressure within the second preset duration, an identifier of the gasbag mounted on the wearable device.

In a possible implementation, the differential pressure sensor includes a first pressure sensor and a second pressure sensor, the first pressure sensor is disposed at the air nozzle, the first pressure sensor is configured to detect a first pressure at the air nozzle, the second pressure sensor is configured to detect a second pressure, the second pressure is an environmental pressure, and the differential pressure at the air nozzle is a difference between the first pressure and the second pressure.

In a possible implementation, the positive pin, the negative pin, and the air nozzle are disposed on an outer surface of the wearable device.

According to a fourth aspect, an embodiment of this application provides a wearable device. The wearable device may include a processor and a memory. The memory is configured to store computer-executable program code, where the program code includes instructions. When the processor executes the instructions, the instructions enable the wearable device to perform the method in the first aspect.

According to a fifth aspect, an embodiment of this application provides a wearable device. The wearable device may be the gasbag detection apparatus for the wearable device in the second aspect or the wearable device in the first aspect. The wearable device may include a unit, module, or circuit configured to perform the method in the first aspect.

According to a sixth aspect, an embodiment of this application provides a computer program product including instructions. When the instructions are run on a computer, the computer is enabled to perform the method in the first aspect.

According to a seventh aspect, an embodiment of this application provides a computer-readable storage medium. The computer-readable storage medium stores instructions. When the instructions are run on a computer, the computer is enabled to perform the method in the first aspect.

For beneficial effects of the possible implementations of the second aspect to the seventh aspect, refer to beneficial effects brought by the first aspect. Details are not described herein again.

Embodiments of this application provide a gasbag detection method and apparatus for a wearable device, and a wearable device. The method includes: when a gasbag is mounted on the wearable device, detecting, based on a wrist circumference of a user, whether the gasbag mounted on the wearable device matches the wrist circumference of the user; and if the gasbag mounted on the wearable device does not match the wrist circumference of the user, outputting first prompt information, where the first prompt information indicates to mount a gasbag that matches the wrist circumference of the user. In this embodiment of this application, the wearable device may detect, based on the wrist circumference of the user, whether the gasbag mounted on the wearable device matches the wrist circumference of the user. When the gasbag mounted on the wearable device does not match the wrist circumference, the user may be prompted to mount the gasbag that matches the wrist circumference of the user, to improve accuracy of blood pressure measurement.

### BRIEF DESCRIPTION OF THE DRAWINGS

FIG. 1 is a schematic diagram of a structure of a wearable device according to an embodiment of this application;

FIG. 2 is a schematic diagram of another structure of a wearable device according to an embodiment of this application;

FIG. 3 is a schematic diagram of detecting a gasbag by using a Hall effect sensor according to an embodiment of this application;

FIG. 4 is a schematic flowchart of an embodiment of a gasbag detection method for a wearable device according to an embodiment of this application;

FIG. 5 is a schematic diagram of an interface according to an embodiment of this application;

FIG. 6 is a schematic diagram of another interface according to an embodiment of this application;

FIG. 7 is a schematic diagram of another structure of a wearable device according to an embodiment of this application;

FIG. 8 is a schematic flowchart of another embodiment of a gasbag detection method for a wearable device according to an embodiment of this application; and

FIG. 9 is a schematic diagram of a structure of a gasbag detection apparatus for a wearable device according to an embodiment of this application.

### DETAILED DESCRIPTION OF ILLUSTRATIVE EMBODIMENTS

A wearable device in embodiments of this application may be, but is not limited to, a device that can be worn on a wrist of a user, like a watch or a band. The wearable device in embodiments of this application is configured to be worn on the wrist of the user, and a gasbag may be disposed on a side that is in contact with the wrist of the user. The gasbag is configured to measure a blood pressure of the user. It should be understood that the gasbag disposed on the wearable device in embodiments of this application is a detachable gasbag, and the wearable device can support gasbags of a plurality of types. Gasbags of different types have different sizes (which may be understood as volumes). In this way, fully inflated gasbags of different types can cover different areas of the wrist.

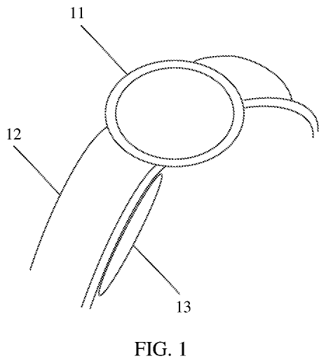

FIG. 1 is a schematic diagram of a structure of a wearable device according to an embodiment of this application. In FIG. 1, an example in which the wearable device is a watch is used for description. As shown in FIG. 1, the watch 10 includes a watch head 11, a watchband 12, and a gasbag 13 disposed on the watchband 12. The watchband 12 may be mounted on the watch head 11, and the watchband 11 is detachable. The gasbag 13 may be mounted on the watchband, and the gasbag 13 is detachable. In other words, a user may mount the gasbag 13 on the watchband 12, or may remove the gasbag 13 from the watchband 12. The watch 10 may support a plurality of types of gasbags 13. In other words, the user may remove one type of gasbag from the watchband 12, and mount another type of gasbag on the watchband 12. In FIG. 1, one gasbag is used as an example for description. It should be understood that the gasbag 13 in FIG. 1 is a gasbag that is not inflated.

In an embodiment, the gasbag 13 may be attached to the watchband 12. For example, a velcro is disposed on the gasbag 13 and the watchband 12, and the gasbag 13 and the watchband 12 may be attached together by using the velcro. Alternatively, in an embodiment, the gasbag 13 may be clamped on the watchband 12. For example, a through hole is disposed in the watchband 12, a buckle is disposed on the gasbag 13, and the buckle may be clamped in the through hole, so that the gasbag 13 is clamped on the watchband 12. In an embodiment, the velcro, the through hole, and the like that are disposed on/in the wearable device are gasbag detachment components. A manner in which the gasbag 13 is disposed on the watchband 12 is not limited in embodiments of this application, that is, the gasbag detachment component disposed on the wearable device is not limited. The two manners are examples for description.

It should be understood that the gasbag 13 is located on a side that is in contact with a wrist of the user. When the user wears the watch 10 on the wrist, the gasbag 13 is in contact with the wrist of the user. When the user uses the watch 10 to measure a blood pressure, the watch 10 may be used to inflate the gasbag 13, to detect the blood pressure of the user. A principle in which the watch 10 is used to inflate the gasbag 13 to detect the blood pressure of the user is not described herein. For details, refer to related descriptions in the conventional technology.

It should be understood that the watchband 12 on which the gasbag 13 is disposed may be referred to as a gasbag watchband. In an embodiment, a common watchband (the common watchband is a watchband on which the gasbag 13 cannot be mounted) may alternatively be mounted on the watch 10. In the following embodiments, the common watchband is referred to as a non-gasbag watchband.

Gasbags of different types have different sizes, and fully inflated gasbags can cover different areas of the wrist of the user. It may be figured out that a larger volume of the gasbag indicates a larger covered area of the wrist of the user, and a more accurate blood pressure measurement result; and a smaller volume of the gasbag indicates more convenient use. Wrists of different users have different thicknesses, namely, different wrist circumferences. Therefore, if a blood pressure of a user needs to be accurately measured, different users need different gasbags, and a result of measuring a blood pressure by using a gasbag of an incorrect type is inaccurate.

For example, for a user with a large wrist circumference, if a small-sized gasbag is used, the gasbag cannot fully cover the wrist of the user, and a blood pressure measurement result is inaccurate. For a user with a small wrist circumference, a large-sized gasbag can fully cover the wrist of the user, and a blood pressure measurement result is accurate. However, for the user with the small wrist circumference, a small-sized gasbag can fully cover the wrist of the user, and the small-sized gasbag is more convenient for use. Therefore, when the user performs blood pressure measurement by using the wearable device, it is important to recommend an appropriate gasbag to the user. This can improve user experience.

In this way, based on the foregoing problem, embodiments of this application provide a gasbag detection method for a wearable device. For the wearable device that supports a plurality of types of gasbags, when a user uses the wearable device to measure a blood pressure, the wearable device may first detect whether a gasbag currently mounted on the wearable device matches a wrist circumference of the user. If the gasbag matches the wrist circumference of the user, the wearable device may directly measure the blood pressure of the user, and accuracy of a blood pressure measurement result is high. If the gasbag does not match the wrist circumference of the user, the wearable device may prompt the user to replace the gasbag with a gasbag that matches the wrist circumference of the user, to improve accuracy of the blood pressure measurement result.

Before the gasbag detection method for a wearable device provided in embodiments of this application is described, a structure of the wearable device provided in embodiments of this application is first described.

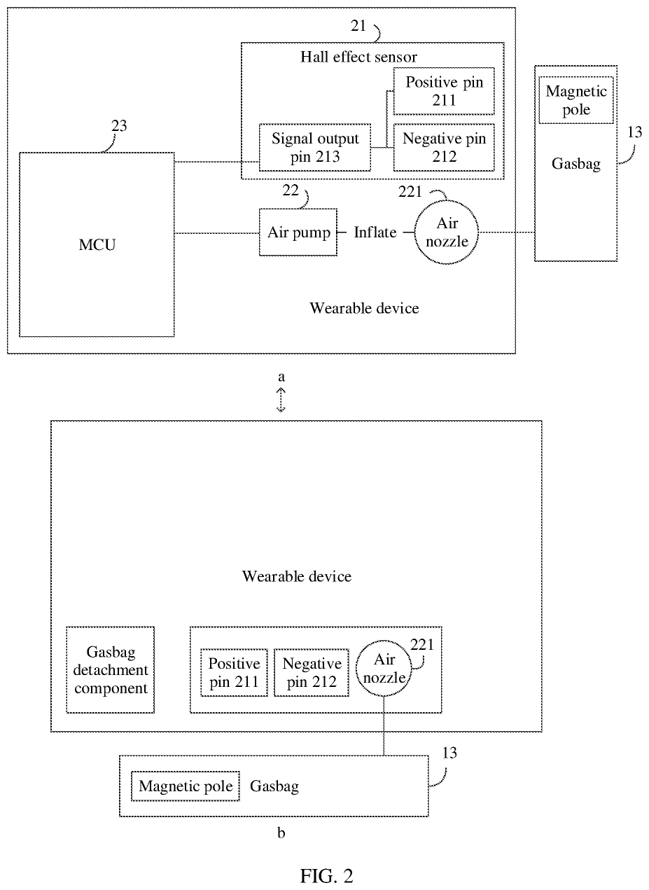

FIG. 2 is a schematic diagram of another structure of a wearable device according to an embodiment of this application. As shown in a in FIG. 2, the wearable device in this embodiment of this application may include a Hall effect sensor 21, an air pump 22, and a micro controller unit (micro controller unit, MCU) 23. The MCU 23 is separately connected to the Hall effect sensor 21 and the air pump 22. In FIG. 2, an example in which the wearable device is a watch is used for description. In an embodiment, the Hall effect sensor 21, the air pump 22, and the MCU 23 are all disposed in a watch head 11 of the watch 10.

The Hall effect sensor 21 includes three pins: a positive (S pole) pin 211, a negative (N pole) pin 212, and a signal output pin 213. In an embodiment, the positive pin 211 and the negative pin 212 on the Hall effect sensor 21 are disposed on an outer surface of the watch head 11, and the signal output pin 213 on the Hall effect sensor 21 is disposed in the watch head 10. The air pump 22 is disposed in the watch head 11, and an air nozzle 221 connected to the air pump 22 is disposed on the outer surface of the watch head 11. It should be understood that, in embodiments of this application, locations at which the positive pin 211, the negative pin 212, and the air nozzle 221 are disposed on the outer surface of the watch head 11 are not limited. In b in FIG. 2, an example in which the positive pin 211, the negative pin 212, and the air nozzle 221 are disposed in parallel on the outer surface of the watch head 11 is used for description. It should be understood that a in FIG. 2 is a schematic diagram of a connection structure of the wearable device, and b in FIG. 2 is a schematic diagram of disposing the positive pin 211, the negative pin 212, and the air nozzle 221 on the outer surface of the wearable device.

It should be understood that, in an embodiment, a gasbag detachment component is disposed on the outer surface of the watch head, and a gasbag 13 may be mounted on the wearable device by using the gasbag detachment component. For the gasbag detachment component, refer to the related descriptions.

An air intake is disposed on the gasbag 13. When the gasbag 13 is mounted on the wearable device, the air nozzle 221 communicates with the air intake of the gasbag 13. When a user measures a blood pressure by using the wearable device, the MCU 23 may control the air pump 22 to blow air through the air nozzle 221, to inflate the gasbag 13. In an embodiment, the MCU 23 may send a pulse width modulation (pulse width modulation, PWM) wave to the air pump, to control the air pump 22 to blow air through the air nozzle 221.

In this embodiment, to cooperate with the positive pin 211 and the negative pin 212 in the wearable device, magnetic poles, for example, a positive magnetic pole (referred to as a positive pole for short below), or a negative magnetic pole (referred to as a negative pole for short below), may be disposed on gasbags of different types, or no positive or negative pole is disposed, to distinguish gasbags of different types (for a specific distinguishing manner, refer to the following related descriptions). It should be understood that a type represents different gasbags. Different types of gasbags may be understood as gasbags with different identifiers, and the identifiers may include but are not limited to a type, a volume, or a number. In the following embodiment, an example in which the identifier is a type is used for description.

It should be understood that the positive pin 211 on the Hall effect sensor 21 can sense the positive pole (S pole), and the negative pin 212 can sense the negative pole (N pole). When the magnetic pole approaches to the positive pin 211 or the negative pin 212, the positive pin 211 or the negative pin 212 can sense the magnetic pole, and the positive pin 211 or the negative pin 212 outputs a high level or a low level through the signal output pin 213 (the high level is used as an example for description below). Correspondingly, the positive pin 211 and the negative pin 212 may output a low level when the magnetic pole is not sensed. For example, when the positive pin 211 senses the positive pole (S pole), the positive pin 211 outputs a high level through the signal output pin 213; and when the negative pin 212 senses the negative pole (N pole), the negative pin 212 outputs a high level through the signal output pin 213. It should be understood that the low level may be understood as a level less than a first threshold, the high level may be understood as a level greater than a second threshold, and the second threshold is greater than the first threshold.

For example, in an embodiment, a first gasbag and a second gasbag in this embodiment of this application are used to represent gasbags of different types. A volume of the first gasbag is less than that of the second gasbag. For example, a negative pole is disposed on the first gasbag, a positive pole is disposed on the second gasbag, and no magnetic pole (a positive pole or a negative pole) is disposed on a non-gasbag watchband.

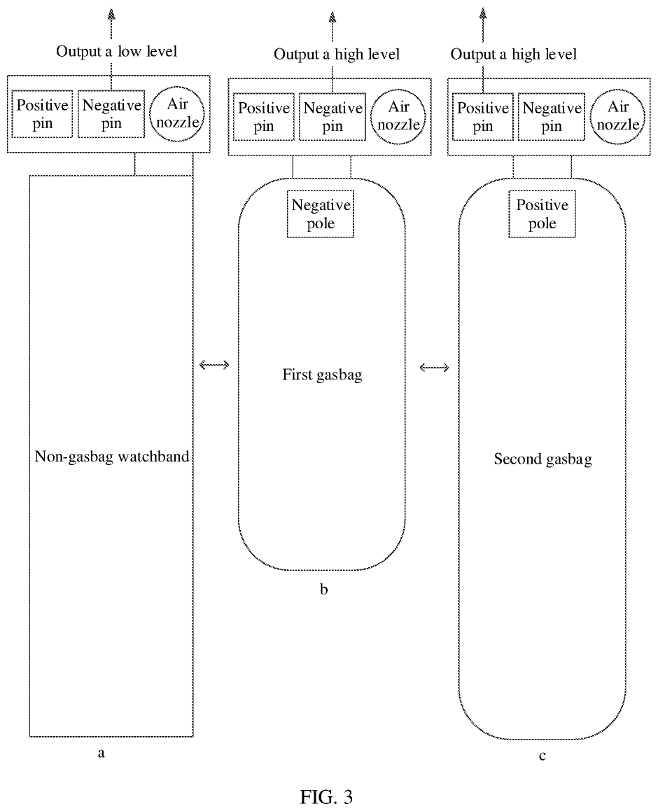

When no non-gasbag watchband is mounted on the watch head, or only a gasbag watchband is mounted on the watch head but no gasbag is mounted, the positive pin 211 and the negative pin 212 on the Hall effect sensor 21 do not sense approaching of the magnetic pole. Therefore, the positive pin 211 and the negative pin 212 may output a low level through the signal output pin 213. As shown in a in FIG. 3, when the user mounts the non-gasbag watchband on the watch head 10, because no magnetic pole is disposed on the non-gasbag watchband, the positive pin 211 and the negative pin 212 on the Hall effect sensor 21 do not sense approaching of the magnetic pole. Therefore, the positive pin 211 and the negative pin 212 may output a low level through the signal output pin 213. In a in FIG. 3, an example in which the negative pin 212 outputs a low level is used for description.

As shown in b in FIG. 3, when the user mounts the first gasbag on the gasbag watchband, and mounts the gasbag watchband on the watch head 11, the negative pole of the first gasbag is in contact with the negative pin 212 on the Hall effect sensor 21, and the negative pin 212 senses the negative pole. Therefore, a high level may be output through the signal output pin 213. As shown in c in FIG. 3, when the user mounts the second gasbag on the gasbag watchband, and mounts the gasbag watchband on the watch head 11, the positive pole of the second gasbag is in contact with the positive pin 211 on the Hall effect sensor 21, and the positive pin 211 senses the positive pole. Therefore, the positive pin 211 may output a high level through the signal output pin 213.

As shown in a in FIG. 2, the MCU 23 is connected to the Hall effect sensor 21, and the MCU 23 may detect the level output through the signal output pin 213 on the Hall effect sensor 21. In this way, the MCU 23 may detect, by detecting the level output through the signal output pin 213 on the Hall effect sensor 21, whether the gasbag is mounted on the wearable device, and detect, based on the positive pin or the negative pin that outputs, through the signal output pin, the level greater than the second threshold, an identifier of the gasbag mounted on the wearable device. In other words, the MCU 23 may detect, by detecting the level (a high level or a low level) output through the signal output pin 213 on the Hall effect sensor 21, whether the gasbag is mounted on the watch, and the MCU 23 may further detect a type of the gasbag by detecting the pin (the positive pin 211 or the negative pin 212) that outputs a high level.

For example, if the MCU 23 detects that a low level is output through the signal output pin 213 on the Hall effect sensor 21, it may be determined that no gasbag is mounted on the watch. When the MCU 23 detects that a high level is output through the signal output pin 213 on the Hall effect sensor 21, it may be determined that the gasbag is mounted on the watch. If the pin that outputs the high level is the positive pin 211, it may be determined that the gasbag mounted on the watch is the second gasbag. If the pin that outputs the high level is the negative pin 212, it may be determined that the gasbag mounted on the watch is the first gasbag.

It should be understood that, a positive pole may be disposed on the first gasbag, and a negative pole may be disposed on the second gasbag. The foregoing is an example for description. The magnetic pole disposed on the first gasbag is different from the magnetic pole disposed on the second gasbag, so that the user can distinguish between the first gasbag and the second gasbag.

Based on FIG. 2 and FIG. 3, the following describes, with reference to a specific embodiment, the gasbag detection method for a wearable device provided in embodiments of this application. The following several embodiments may be combined with each other, and a same or similar concept or process may not be described repeatedly in some embodiments.

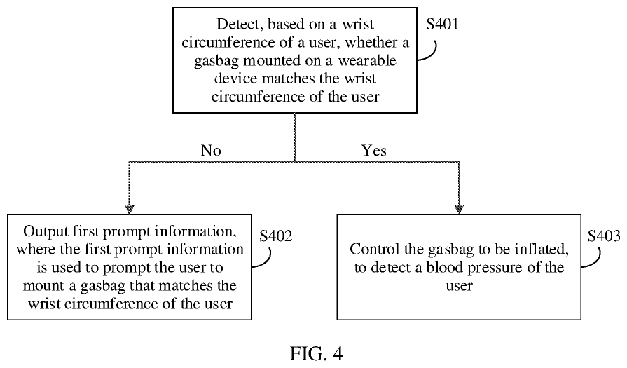

FIG. 4 is a schematic flowchart of an embodiment of a gasbag detection method for a wearable device according to an embodiment of this application. As shown in FIG. 4, the gasbag detection method for a wearable device provided in embodiments of this application may include the following steps.

S401: The wearable device detects, based on a wrist circumference of a user, whether a gasbag mounted on the wearable device matches the wrist circumference of the user. If the gasbag mounted on the wearable device does not match the wrist circumference of the user, S402 is performed; or if the gasbag mounted on the wearable device matches the wrist circumference of the user, S403 is performed.

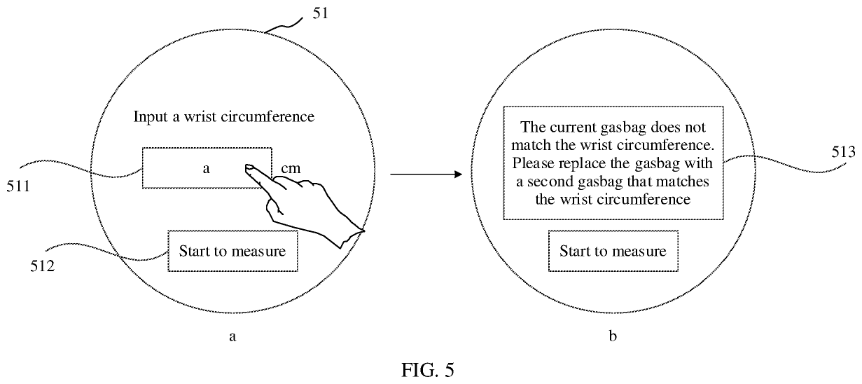

The wearable device provides a blood pressure measurement service for the user. When the user measures a blood pressure by using the wearable device, the user may input the wrist circumference of the user on an interface displayed on the wearable device or on an interface displayed on an electronic device (like a mobile phone or a tablet) bound to the wearable device. FIG. 5 is a schematic diagram of an interface according to an embodiment of this application. In FIG. 5, an example in which the user inputs the wrist circumference on the interface displayed on the wearable device is used for description. As shown in a in FIG. 5, an interface 51 of the wearable device displays an input box 511 and a measurement control 512 of the wrist circumference of the user.

When the user uses a blood pressure measurement function of the wearable device, that is, when the user has not input the wrist circumference, the wearable device may further output prompt information to prompt the user how to measure the wrist circumference. Details are not described herein. For details, refer to conventional related descriptions. The user may input the wrist circumference in the input box. It should be understood that FIG. 5 is an example in which the user inputs the wrist circumference on the interface displayed on the wearable device. The user may alternatively input the wrist circumference of the user to the wearable device in a voice or another manner.

In an embodiment, in response to detecting that the user inputs the wrist circumference in the input box 511, that is, detecting an instruction indicating that the user inputs the wrist circumference, the wearable device may detect, based on the wrist circumference of the user, whether the gasbag mounted on the wearable device matches the wrist circumference. In this embodiment, S401 may be replaced with S401A. S401A: In response to detecting the instruction indicating that the user inputs the wrist circumference, the wearable device detects, based on the wrist circumference of the user, whether the gasbag mounted on the wearable device matches the wrist circumference. If the gasbag mounted on the wearable device does not match the wrist circumference of the user, S402 is performed; or if the gasbag mounted on the wearable device matches the wrist circumference of the user, S403 is performed.

The wearable device may detect a type of the gasbag mounted on the wearable device, to detect whether the type of the gasbag matches the wrist circumference of the user. A manner in which the wearable device detects the type of the gasbag mounted on the wearable device may be as follows: The wearable device may detect, by detecting a level (a high level or a low level) output by the Hall effect sensor, whether the gasbag is mounted on the watch, and may further detect the type of the gasbag by detecting a pin that outputs a high level. For details, refer to the related descriptions in FIG. 2 and FIG. 3. In this embodiment, different magnetic poles are disposed on gasbags mounted on the wearable device. For example, a negative pole is disposed on a first gasbag, and a positive pole is disposed on a second gasbag.

In an embodiment, the wearable device may store a mapping relationship between a wrist circumference and a type of a gasbag, and the mapping relationship represents a type of a gasbag that matches each wrist circumference. For example, the mapping relationship may be shown in Table 1.

TABLE 1

| Wrist circumference (cm) | Type of the gasbag |
|---|---|
| a to b | First gasbag |
| b to c | Second gasbag |

After obtaining the type of the gasbag mounted on the wearable device, the wearable device may detect, based on the mapping relationship between the wrist circumference and the type of the gasbag, whether the gasbag mounted on the wearable device is the gasbag mapped to the wrist circumference. If the gasbag mounted on the wearable device is the gasbag mapped to the wrist circumference, it is determined that the gasbag mounted on the wearable device matches the wrist circumference, or if the gasbag mounted on the wearable device is not the gasbag mapped to the wrist circumference, it is determined that the gasbag mounted on the wearable device does not match the wrist circumference.

For example, if the wrist circumference of the user falls within a range from a cm to b cm, and the wearable device detects that the gasbag mounted on the wearable device is the second gasbag, or no gasbag is mounted on the wearable device, the wearable device may determine that the gasbag mounted on the wearable device does not match the wrist circumference. If the wearable device detects that the gasbag mounted on the wearable device is the first gasbag, the wearable device may determine that the gasbag mounted on the wearable device matches the wrist circumference.

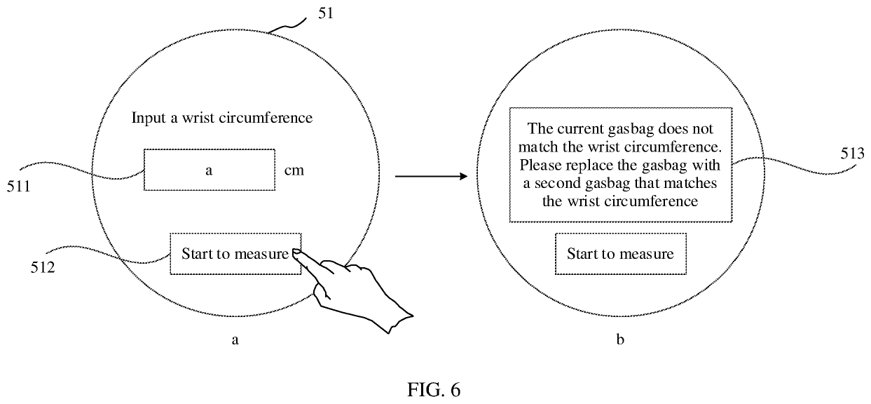

In an embodiment, when the user has used the blood pressure measurement function, that is, the user has input the wrist circumference in the input box 511, in this embodiment, as shown in a in FIG. 6, the user may directly operate the measurement control 512 without inputting the wrist circumference, to trigger the wearable device to start to measure the blood pressure of the user. In response to detecting that the user operates the measurement control 512, that is, detecting that the user inputs an instruction for measuring the blood pressure, the wearable device may detect, based on the wrist circumference of the user, whether the gasbag mounted on the wearable device matches the wrist circumference. Specifically, the wearable device may detect, based on a wrist circumference that is previously input by the user, whether the gasbag mounted on the wearable device matches the wrist circumference.

Correspondingly, in this embodiment, S401 may be replaced with S401B. S401B: In response to detecting that the user inputs the instruction for measuring the blood pressure, the wearable device detects, based on the wrist circumference of the user, whether the gasbag mounted on the wearable device matches the wrist circumference. If the gasbag mounted on the wearable device does not match the wrist circumference of the user, S402 is performed; or if the gasbag mounted on the wearable device matches the wrist circumference of the user, S403 is performed.

The wearable device detects, based on the wrist circumference of the user, whether the gasbag mounted on the wearable device matches the wrist circumference, which may refer to the related descriptions.

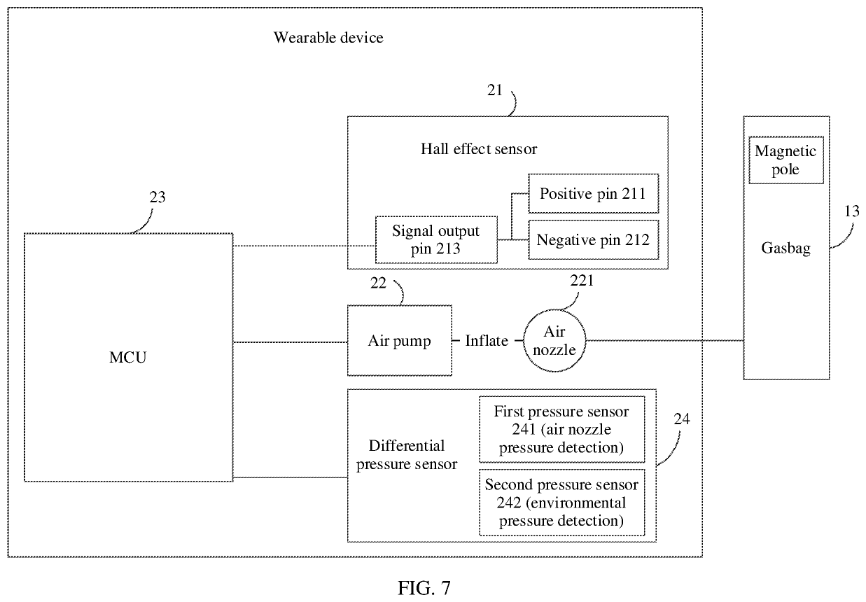

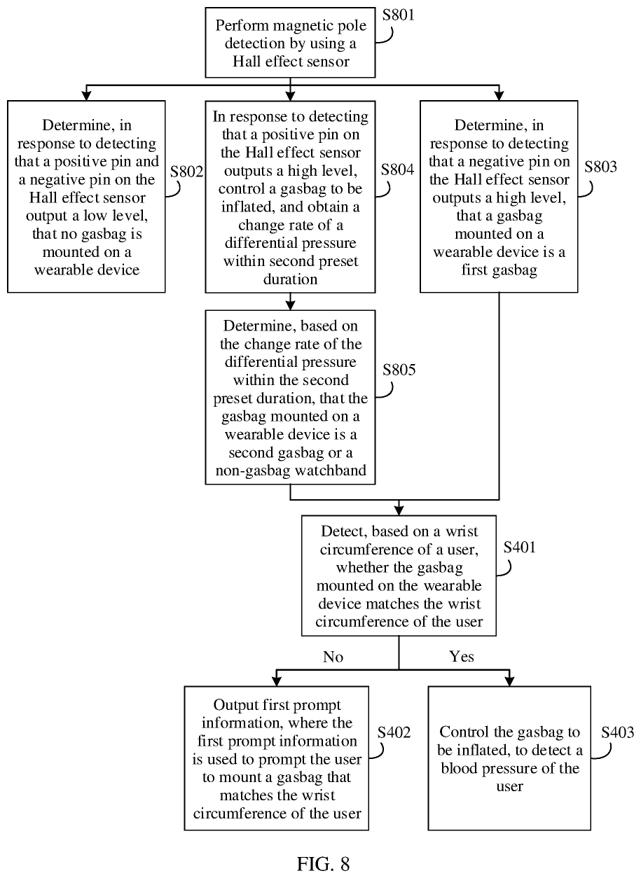

It should be understood that before detecting whether the gasbag mounted on the wearable device matches the wrist circumference, the wearable device may first detect whether the gasbag is mounted on the wearable device. When the gasbag is mounted on the wearable device, the wearable device may detect whether the gasbag mounted on the wearable device matches the wrist circumference. The wearable device detects whether the gasbag is mounted on the wearable device, which may refer to the related descriptions of FIG. 2, FIG. 3, and FIG. 7, and related descriptions of FIG. 8.

S402: The wearable device outputs first prompt information, where the first prompt information is used to prompt the user to mount the gasbag that matches the wrist circumference of the user.

As shown in a in FIG. 5, after the user inputs the wrist circumference in the input box 511, if the wearable device detects that the gasbag mounted on the wearable device does not match the wrist circumference, the wearable device may output the first prompt information. The first prompt information is used to prompt the user to mount the gasbag that matches the wrist circumference of the user, and the first prompt information may include an identifier of the gasbag that matches the wrist circumference of the user. The identifier of the gasbag that matches the wrist circumference of the user may be information used to distinguish the gasbag, such as a type of the gasbag or a volume of the gasbag.

In an embodiment, a manner in which the wearable device outputs the first prompt information may be: The wearable device outputs the first prompt information in a voice broadcast manner, or displays the first prompt information on the interface displayed on the wearable device. A manner in which the wearable device outputs the first prompt information is not limited in embodiments of this application. As shown in b in FIG. 5, the manner in which the wearable device displays the first prompt information on the interface is used as an example for description. For example, the wearable device may display, on the interface 51 displayed on the wearable device, a prompt box 513 including the first prompt information, where the prompt box 513 displays “The current gasbag does not match the wrist circumference. Please replace the current gasbag with the second gasbag (the large-sized gasbag) that matches the wrist circumference”. Similarly, if the wearable device detects that the gasbag mounted on the wearable device does not match the wrist circumference, as shown in b in FIG. 6, the wearable device may display, on the interface 51, the prompt box 513 including the first prompt information.

In this embodiment, the user may remove the gasbag that does not match the wrist circumference of the user, and mount the wearable device that matches the wrist circumference of the user. After mounting the wearable device that matches the wrist circumference of the user, the user may operate the measurement control 512 to continue to trigger the wearable device to start to measure the blood pressure. In this way, the wearable device may perform S401B, to determine that the gasbag mounted on the wearable device matches the wrist circumference, and then perform S403.

S403: The wearable device controls the gasbag to be inflated, to detect the blood pressure of the user.

In an embodiment, the wearable device may control an air pump to start working, so that the air pump may inflate the gasbag through an air nozzle. A process in which the wearable device inflates the gasbag and a process of detecting the blood pressure of the user are not described in this embodiment of this application. For details, refer to related descriptions in the conventional technology.

FIG. 5 is used as an example. After the user mounts the wearable device that matches the wrist circumference of the user, the wearable device may perform S403. After measuring the blood pressure of the user, the wearable device may display a blood pressure measurement result on an interface of the wearable device, which is not shown in FIG. 5.

In this embodiment of this application, when the user inputs the wrist circumference to the wearable device or triggers the wearable device to measure the blood pressure, the wearable device may detect, based on the wrist circumference of the user, whether the gasbag mounted on the wearable device matches the wrist circumference of the user. When the gasbag mounted on the wearable device does not match the wrist circumference, the user may be prompted to mount the gasbag that matches the wrist circumference of the user, to improve accuracy of blood pressure measurement.

In the embodiment, the wearable device detects the type of the gasbag by detecting the level output by the Hall effect sensor disposed in the wearable device. The type of the gasbag is detected by the Hall effect sensor based on the level output by the positive pin 211 or the negative pin 212 on the Hall effect sensor 21, and the type of the gasbag that can be identified by the Hall effect sensor is limited, for example, the second gasbag due to which the positive pin 211 outputs the high level, and the first gasbag due to which the negative pin 212 outputs the high level. Consequently, an application range is small, and the type of the gasbag supported by the wearable device is limited.

As shown in FIG. 2 and FIG. 7, in an embodiment, compared with FIG. 2, a differential pressure sensor 24 is further disposed on the wearable device, and the MCU 23 may be connected to the differential pressure sensor 24. In an embodiment, the MCU 23 may be connected to the differential pressure sensor 24 through an inter-integrated circuit (inter-integrated circuit, I2C) bus. The differential pressure sensor 24 is configured to detect a differential pressure at the air nozzle 221.

In this embodiment, in response to receiving an instruction indicating that the user inputs a wrist circumference or an instruction that is input by the user and for measuring blood pressure, the MCU 23 may control the air pump 22 to blow air into the gasbag at a preset rate through the air nozzle 221, to detect the type of the gasbag based on a change rate of the differential pressure of pressures collected by a first pressure sensor 241 and a second pressure sensor 242. For details, refer to the following descriptions.

The differential pressure sensor 24 may include the first pressure sensor 241 and the second pressure sensor 242. In an embodiment, the first pressure sensor 241 is disposed at the air nozzle 221, and the second pressure sensor 242 communicates with an external environment. Specifically, the MCU 23 may be separately connected to the first pressure sensor 241 and the second pressure sensor 242. The first pressure sensor 241 is configured to detect a first pressure at the air nozzle 221, and the second pressure sensor 242 is configured to detect an environmental pressure, where the environmental pressure is a second pressure.

In a process in which the air pump 22 blows air to the gasbag through the air nozzle 221 at the preset rate, the first pressure sensor 241 is configured to detect once the first pressure at a first location at an interval of first preset duration. For example, the first preset duration is 5 ms. The second pressure sensor 242 is configured to detect once the second pressure at a second location at the interval of first preset duration. It should be understood that the first location is a location of the first pressure sensor 241, and the second location is a location of the second pressure sensor 242.

In other words, within second preset duration, the first pressure sensor 241 may collect the first pressure N1 times, and the second pressure sensor 242 may collect the second pressure N1 times. N1 is an integer greater than or equal to 1.

The MCU 23 may obtain an average value of first pressures collected by the first pressure sensor 241 within the second preset duration, namely, a first pressure average value, or may obtain an average value of second pressures collected by the second pressure sensor 242 within the second preset duration, namely, a second pressure average value. For example, the second preset duration may be 1s.

The MCU 23 obtains a change rate of the differential pressure within the second preset duration based on the first pressure average value and the second pressure average value. For example, the MCU 23 obtains the change rate of the differential pressure within the second preset duration, which may refer to Formula 1.

$${V}_{p} = \frac{{P}_{1} - {P}_{2}}{T} \qquad Formula 1$$

Herein, V_{P} is the change rate of the differential pressure, P₁ is the first pressure average value, P₂ is the second pressure average value, and I is the second preset duration.

The MCU 23 may detect the type of the gasbag based on the change rate of the differential pressure within the second preset duration. For details, refer to related descriptions in the following embodiment.

Based on the structure of the wearable device in FIG. 7, in this embodiment, because the MCU 23 is included in the wearable device, the following uses an example in which the wearable device is an execution body for description.

In an embodiment, the wearable device stores a mapping relationship between a change rate of a differential pressure and a type of a gasbag. It may be understood that a smaller volume of the gasbag indicates a larger change rate of the differential pressure within the second preset duration. When the non-gasbag watchband is disposed on the wearable device, the non-gasbag watchband blocks the air nozzle 221. In this case, in the process in which the air pump 22 blows air to the gasbag through the air nozzle 221 at the preset rate, a large differential pressure is generated in short time. Therefore, the change rate of the differential pressure is large in the short time. When no non-gasbag watchband is mounted on the wearable device, or no gasbag is mounted on the wearable device, the air nozzle 221 communicates with air. Therefore, the change rate of the differential pressure within the second preset duration is o.

In other words, if the change rate of the differential pressure within the second preset duration is 0, or the change rate of the differential pressure within the second preset duration is greater than or equal to a first change rate threshold, the wearable device determines that no gasbag is mounted on the wearable device. If the change rate of the differential pressure within the second preset duration is less than the first change rate threshold, the wearable device determines that the gasbag is mounted on the wearable device. The first change rate threshold may be a predefined value.

In an embodiment, the mapping relationship between the change rate of the differential pressure and the type of the gasbag may be shown in Table 2.

TABLE 2

| Change rate of the | |
|---|---|
| differential pressure | Type of the gasbag |
| a1 to b1 | First gasbag |
| b1 to c1 | Second gasbag |
| c1 to d1 | Third gasbag |
| ≥e | Non-gasbag watchband |
| o | No gasbag or non-gasbag watchband |

In this embodiment of this application, in response to information that the change rate of the differential pressure within the second preset duration is less than the first change rate threshold, the wearable device may detect the type of the gasbag based on the change rate of the differential pressure within the second preset duration and the mapping relationship between the change rate of the differential pressure and the type of the gasbag. After obtaining the type of the gasbag, the wearable device may detect whether the gasbag matches a wrist circumference of the user. For details, refer to the related descriptions in the foregoing embodiments.

In this embodiment of this application, the differential pressure sensor is disposed on the wearable device. The wearable device may blow air by using the air pump, detect the change rate of the differential pressure within the second preset duration by using the differential pressure sensor, and detect the type of the gasbag based on the change rate of the differential pressure and the mapping relationship between the change rate of the differential pressure and the type of the gasbag. After obtaining the type of the gasbag, the wearable device may detect whether the gasbag matches the wrist circumference of the user. In this embodiment, the type of the gasbag may be detected based on the change rate of the differential pressure. This is applicable to a plurality of types of gasbags, and is not limited to two gasbags (in Table 2, three types of gasbags are used as an example for description). An application range is wide. When the gasbag mounted on the wearable device does not match the wrist circumference, the user may still be prompted to mount a gasbag that matches the wrist circumference of the user, to improve accuracy of blood pressure measurement.

Based on the structure of the wearable device shown in FIG. 7, in an embodiment, the wearable device may combine a manner of detecting the type of the gasbag by using the Hall effect sensor with a manner of detecting the type of the gasbag based on the change rate of the differential pressure, to detect whether the gasbag matches the wrist circumference of the user.

In this embodiment, a same magnetic pole may be disposed on gasbags mounted on the wearable device, or at least one gasbag is disposed with a same magnetic pole as the non-gasbag watchband. In this embodiment, to accurately detect whether the gasbag is mounted on the wearable device and the type of the gasbag mounted on the wearable device, the manner of detecting the type of the gasbag by using the Hall effect sensor needs to be combined with the manner of detecting the type of the gasbag based on the change rate of the differential pressure.

In other words, the wearable device may detect, based on the level output through the signal output pin and the change rate of the differential pressure within the second preset duration, whether the gasbag is mounted on the wearable device, and the wearable device may detect, based on the pin (the positive pin or the negative pin) that outputs, through the signal output pin, the level greater than the second threshold and the change rate of the differential pressure within the second preset duration, the identifier (type) of the gasbag mounted on the wearable device.

In an embodiment, the negative pole is disposed on the first gasbag, the positive pole is disposed on the second gasbag, and the positive pole is disposed on the non-gasbag watchband.

As shown in FIG. 8, in this embodiment, the gasbag detection method for a wearable device provided in embodiments of this application may include the following steps.

S801: The wearable device performs magnetic pole detection by using the Hall effect sensor.

That the wearable device performs magnetic pole detection by using the Hall effect sensor may be understood as follows: The wearable device detects whether the positive pin or the negative pin on the Hall effect sensor outputs the high level, so that when detecting that the pin outputs the high level, the wearable device can obtain a magnetic pole detected by the pin that outputs the high level.

In this embodiment, the negative pole is disposed on the first gasbag, the positive pole is disposed on the second gasbag, and the positive pole is disposed on the non-gasbag watchband. When no non-gasbag watchband is mounted on the wearable device, or only the gasbag watchband is mounted on the wearable device, but no gasbag is mounted, the positive pin and the negative pin on the Hall effect sensor do not detect approaching of the magnetic pole. Therefore, the positive pin and the negative pin may output a low level through the signal output pin.

When the user mounts the first gasbag on the gasbag watchband, and mounts the gasbag watchband on the wearable device, the negative pole of the first gasbag is in contact with the negative pin on the Hall effect sensor, and the negative pin detects the negative pole. Therefore, a high level may be output through the signal output pin. When the user mounts the second gasbag on the gasbag watchband and mounts the gasbag watchband on the watch head, the positive pole of the second gasbag is in contact with the positive pin on the Hall effect sensor, and the positive pin detects the positive pole. Therefore, a high level may be output through the signal output pin. Because the positive pole is disposed on the non-gasbag watchband, when the user mounts the gasbag watchband on the watch head, the positive pole of the non-gasbag watchband is in contact with the positive pin on the Hall effect sensor, and the positive pin detects the positive pole. Therefore, a high level may be output through the signal output pin.

In an embodiment, S801 may be replaced with S801A or S801B.

S801A: The wearable device performs, in response to detecting the instruction indicating that the user inputs the wrist circumference, magnetic pole detection by using the Hall effect sensor.

S801B: The wearable device performs, in response to detecting that the user inputs the instruction for measuring the blood pressure, magnetic pole detection by using the Hall effect sensor.

S802: The wearable device determines, in response to detecting that the positive pin and the negative pin on the Hall effect sensor output a low level, that no gasbag is mounted on the wearable device.

As described in S801, in response to detecting that the positive pin and the negative pin on the Hall effect sensor output a low level, the wearable device may determine that the non-gasbag watchband is mounted on the wearable device, or that only the gasbag watchband is mounted on the wearable device, and no gasbag is mounted on the wearable device, that is, no gasbag is mounted on the wearable device.

In an embodiment, when determining that no gasbag is mounted on the wearable device, the wearable device may output second prompt information. The second prompt information is used to prompt the user to mount the gasbag. After the user mounts the gasbag, the wearable device may return to perform S801.

S803: The wearable device determines, in response to detecting that the negative pin on the Hall effect sensor outputs a high level, that the gasbag mounted on the wearable device is the first gasbag.

As described in S801, the wearable device determines, in response to detecting that the negative pin on the Hall effect sensor outputs a high level, that the gasbag mounted on the wearable device is the first gasbag.

S804: In response to detecting that the positive pin on the Hall effect sensor outputs a high level, the wearable device controls the gasbag to be inflated, and obtains the change rate of the differential pressure within the second preset duration.

Because the positive pole is disposed on both the second gasbag and the non-gasbag watchband, when the user mounts the second gasbag or the non-gasbag watchband on the wearable device, the positive pin on the Hall effect sensor outputs a high level. In this way, when detecting that the positive pin on the Hall effect sensor outputs a high level, the wearable device may determine that the second gasbag or the non-gasbag watchband is mounted on the wearable device, but cannot determine whether the second gasbag or the non-gasbag watchband is specifically mounted on the wearable device. Therefore, the wearable device needs to determine, with reference to the detection manner based on the change rate of the differential pressure, whether the second gasbag or the non-gasbag watchband is specifically mounted on the wearable device.

Therefore, in this embodiment, in response to detecting that the positive pin on the Hall effect sensor outputs a high level, the wearable device controls the gasbag to be inflated, and obtains the change rate of the differential pressure within the second preset duration. The wearable device controls the gasbag to be inflated, and obtains the change rate of the differential pressure within the second preset duration, which may refer to the related descriptions in the foregoing embodiments.

S805: The wearable device determines, based on the change rate of the differential pressure within the second preset duration, that the gasbag mounted on the wearable device is the second gasbag or the non-gasbag watchband.

For example, the wearable device may determine, based on the mapping relationship (as shown in Table 2) between the change rate of the differential pressure and the type of the gasbag, that the gasbag mounted on the wearable device is the second gasbag or the non-gasbag watchband.

One of S802, S803, and S804 and S805 is selected and performed, and S401 to S403 may be performed after S803 and S805. Because the type of the gasbag is detected and obtained in this embodiment of this application, for performing S401, “The wearable device detects, based on a wrist circumference of a user, whether a gasbag mounted on the wearable device matches the wrist circumference” in S401 is performed. For details, refer to the related descriptions in the foregoing embodiments.

In this embodiment of this application, the wearable device may accurately detect, with reference to the magnetic pole detection manner based on the Hall effect sensor and the detection manner based on the change rate of the pressure, the type of the gasbag disposed on the wearable device, to detect whether the gasbag disposed on the wearable device matches the wrist circumference of the user. When the gasbag mounted on the wearable device does not match the wrist circumference, the user may be prompted to mount the gasbag that matches the wrist circumference of the user, to improve accuracy of blood pressure measurement.

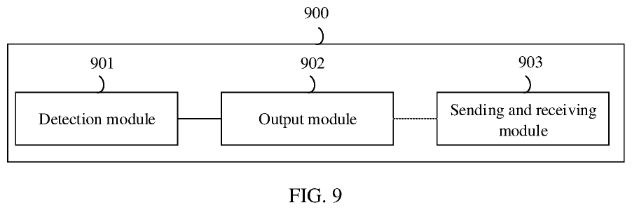

FIG. 9 is a schematic diagram of a structure of a gasbag detection apparatus according to an embodiment of this application. The gasbag detection apparatus in this embodiment may be the wearable device, or may be a chip applied to the wearable device. The gasbag detection apparatus may be configured to perform actions of the wearable device in the foregoing method embodiments. As shown in FIG. 9, the gasbag detection apparatus 900 may include a detection module 901, an output module 902, and a sending and receiving module 903.

The detection module 901 is configured to: when a gasbag is mounted on the wearable device, detect, based on a wrist circumference of a user, whether the gasbag mounted on the wearable device matches the wrist circumference of the user.

The output module 902 is configured to: if the gasbag mounted on the wearable device does not match the wrist circumference of the user, output first prompt information, where the first prompt information indicates to mount a gasbag that matches the wrist circumference of the user.

In a possible implementation, the detection module 901 is further configured to detect whether the gasbag is mounted on the wearable device.

The detection module 901 is specifically configured to: in response to detecting that the gasbag is mounted on the wearable device, detect an identifier of the gasbag mounted on the wearable device, and detect, based on a mapping relationship between a wrist circumference and an identifier of a gasbag, whether the identifier of the gasbag mounted on the wearable device is an identifier of a gasbag mapped to the wrist circumference of the user.

In a possible implementation, the detection module 901 is specifically configured to: if the identifier of the gasbag mounted on the wearable device is not the identifier of the gasbag mapped to the wrist circumference of the user, determine that the gasbag mounted on the wearable device does not match the wrist circumference of the user; or if the identifier of the gasbag mounted on the wearable device is the identifier of the gasbag mapped to the wrist circumference of the user, determine that the gasbag mounted on the wearable device matches the wrist circumference of the user.

In a possible implementation, the wearable device includes a Hall effect sensor, and the Hall effect sensor includes a positive pin, a negative pin, and a signal output pin.

In response to sensing a positive magnetic pole, the positive pin outputs, through the signal output pin, a level greater than a second threshold; in response to sensing a negative magnetic pole, the negative pin outputs, through the signal output pin, the level greater than the second threshold; when the positive pin does not sense the positive magnetic pole, the positive pin outputs, through the signal output pin, a level less than a first threshold, where the second threshold is greater than the first threshold; and when the negative pin does not sense the negative magnetic pole, the negative pin outputs, through the signal output pin, the level less than the first threshold.

In a possible implementation, the wearable device includes at least two gasbags, magnetic poles are disposed on the gasbags of the at least two gasbags, and a different magnetic pole is disposed on each gasbag.

The detection module 901 is specifically configured to: detect, based on the level output through the signal output pin, whether the gasbag is mounted on the wearable device; and detect, based on the pin that outputs, through the signal output pin, the level greater than the second threshold, the identifier of the gasbag mounted on the wearable device.

In a possible implementation, the at least two gasbags include a first gasbag and a second gasbag, the negative magnetic pole is disposed on the first gasbag, and the positive magnetic pole is disposed on the second gasbag.

The detection module 901 is specifically configured to: if the level output through the signal output pin is less than the first threshold, determine that no gasbag is mounted on the wearable device; or if the level output through the signal output pin is greater than the second threshold, determine that the gasbag is mounted on the wearable device.

In a possible implementation, if the positive pin outputs, through the signal output pin, the level greater than the second threshold, it is determined that the gasbag mounted on the wearable device is the second gasbag; or if the negative pin outputs, through the signal output pin, the level greater than the second threshold, the detection module 901 is specifically configured to determine that the gasbag mounted on the wearable device is the first gasbag.

In a possible implementation, the wearable device includes a differential pressure sensor and a gasbag watchband, the differential pressure sensor is configured to detect a differential pressure at an air nozzle of an air pump in the wearable device, the gasbag is mounted on the gasbag watchband, and when the gasbag watchband on which the gasbag is mounted is mounted on the wearable device, an air intake on the gasbag communicates with the air nozzle.

The detection module 901 is specifically configured to: control the air pump to blow air at a preset rate; obtain a change rate of the differential pressure within second preset duration based on the differential pressure at the air nozzle collected by the differential pressure sensor; detect, based on the change rate of the differential pressure within the second preset duration, whether the gasbag is mounted on the wearable device; and detect, based on the change rate of the differential pressure within the second preset duration, the identifier of the gasbag mounted on the wearable device.

In a possible implementation, the wearable device further includes a non-gasbag watchband, and when the non-gasbag watchband is mounted on the wearable device, the non-gasbag watchband blocks the air nozzle of the air pump.

If the change rate of the differential pressure within the second preset duration is o, or the change rate of the differential pressure within the second preset duration is greater than or equal to a first change rate threshold, the detection module 901 is specifically configured to determine that no gasbag is mounted on the wearable device; or

if the change rate of the differential pressure within the second preset duration is less than the first change rate threshold, the detection module 901 is specifically configured to determine that the gasbag is mounted on the wearable device.

In a possible implementation, the detection module 901 is specifically configured to: in response to information that the change rate of the differential pressure within the second preset duration is less than the first change rate threshold, detect, based on the change rate of the differential pressure within the second preset duration and a mapping relationship between a change rate of a differential pressure and an identifier of a gasbag, the identifier of the gasbag mounted on the wearable device.

In a possible implementation, the wearable device includes: a differential pressure sensor, a gasbag watchband, and at least two gasbags, the differential pressure sensor is configured to detect a differential pressure at an air nozzle of an air pump in the wearable device, the gasbag is mounted on the gasbag watchband, when the gasbag watchband on which the gasbag is mounted is mounted on the wearable device, an air intake on the gasbag communicates with the air nozzle, and magnetic poles disposed on at least two of the following are the same: a first gasbag in the at least two gasbags, a second gasbag in the at least two gasbags, and a non-gasbag watchband.

The detection module 901 is specifically configured to: control the air pump to blow air at a preset rate; obtain a change rate of the differential pressure within second preset duration based on the differential pressure at the air nozzle collected by the differential pressure sensor; detect, based on the level output through the signal output pin and the change rate of the differential pressure within the second preset duration, whether the gasbag is mounted on the wearable device; and detect, based on the pin that outputs, through the signal output pin, the level greater than the second threshold and the change rate of the differential pressure within the second preset duration, the identifier of the gasbag mounted on the wearable device.

In a possible implementation, the negative magnetic pole is disposed on the first gasbag, the positive magnetic pole is disposed on the second gasbag, and the positive magnetic pole is disposed on the non-gasbag watchband.

The detection module 901 is specifically configured to: if the level output through the signal output pin is less than the first threshold, determine that no gasbag is mounted on the wearable device; if the level output through the signal output pin is greater than the second threshold, and the negative pin outputs, through the signal output pin, the level greater than the second threshold, determine that the gasbag mounted on the wearable device is the first gasbag; or if the level output through the signal output pin is greater than the second threshold, and the positive pin outputs, through the signal output pin, the level greater than the second threshold, detect, based on the change rate of the differential pressure within the second preset duration, that the second gasbag or the non-gasbag watchband is mounted on the wearable device.

In a possible implementation, when the non-gasbag watchband is mounted on the wearable device, the non-gasbag watchband blocks the air nozzle of the air pump.

The detection module 901 is specifically configured to: if the change rate of the differential pressure within the second preset duration is greater than or equal to a first change rate threshold, determine that the non-gasbag watchband is mounted on the wearable device; or if the change rate of the differential pressure within the second preset duration is less than the first change rate threshold, determine that the gasbag mounted on the wearable device is the second gasbag.

In a possible implementation, the differential pressure sensor includes a first pressure sensor and a second pressure sensor, the first pressure sensor is configured to detect a first pressure at the air nozzle, the second pressure sensor is configured to detect a second pressure, the second pressure is an environmental pressure, and the differential pressure at the air nozzle is a difference between the first pressure and the second pressure.

The detection module 901 is specifically configured to: obtain once the first pressure collected by the first pressure sensor at an interval of first preset duration; obtain once the second pressure collected by the second pressure sensor at the interval of first preset duration; obtain a first pressure average value based on first pressures collected within the second preset duration; obtain a second pressure average value based on second pressures collected within the second preset duration; and obtain the change rate of the differential pressure within the second preset duration based on the first pressure average value and the second pressure average value.

In a possible implementation, the sending and receiving module 903 is configured to receive an instruction indicating that the user inputs the wrist circumference, or receive an instruction that is input by the user and that is for measuring a blood pressure.

In a possible implementation, the first prompt information includes the identifier of the gasbag that matches the wrist circumference of the user.

The gasbag detection apparatus provided in this embodiment of this application may perform actions of the wearable device in the foregoing method embodiments. Implementation principles and technical effects thereof are similar. Details are not described herein again.

In an embodiment, an embodiment of this application further provides a wearable device, including a Hall effect sensor. The Hall effect sensor includes a positive pin, a negative pin, and a signal output pin.

In response to sensing a positive magnetic pole, the positive pin outputs, through the signal output pin, a level greater than a second threshold; in response to sensing a negative magnetic pole, the negative pin outputs, through the signal output pin, the level greater than the second threshold; when the positive pin does not sense the positive magnetic pole, the positive pin outputs, through the signal output pin, a level less than a first threshold, where the second threshold is greater than the first threshold; and when the negative pin does not sense the negative magnetic pole, the negative pin outputs, through the signal output pin, the level less than the first threshold.

In a possible implementation, the wearable device includes at least two gasbags and a microprocessor unit MCU, and the MCU is connected to the Hall effect sensor; and the MCU is configured to detect the level output through the signal output pin.

In a possible implementation, magnetic poles are disposed on the gasbags of the at least two gasbags, and a different magnetic pole is disposed on each gasbag.

The MCU is further configured to: detect, based on the level output through the signal output pin, whether the gasbag is mounted on the wearable device; and detect, based on the pin that outputs, through the signal output pin, the level greater than the second threshold, an identifier of the gasbag mounted on the wearable device.

In a possible implementation, the wearable device further includes a differential pressure sensor and a gasbag watchband, the differential pressure sensor is configured to detect a differential pressure at an air nozzle of an air pump in the wearable device, the gasbag is mounted on the gasbag watchband, and when the gasbag watchband on which the gasbag is mounted is mounted on the wearable device, an air intake on the gasbag communicates with the air nozzle.

The MCU is further configured to: control the air pump to blow air at a preset rate; obtain a change rate of the differential pressure within second preset duration based on the differential pressure at the air nozzle collected by the differential pressure sensor; detect, based on the change rate of the differential pressure within the second preset duration, whether the gasbag is mounted on the wearable device; and detect, based on the change rate of the differential pressure within the second preset duration, the identifier of the gasbag mounted on the wearable device.

In a possible implementation, the wearable device further includes: a differential pressure sensor, a gasbag watchband, and the at least two gasbags, the differential pressure sensor is configured to detect a differential pressure at an air nozzle of an air pump in the wearable device, the gasbag is mounted on the gasbag watchband, when the gasbag watchband on which the gasbag is mounted is mounted on the wearable device, an air intake on the gasbag communicates with the air nozzle, and magnetic poles disposed on at least two of the following are the same: a first gasbag in the at least two gasbags, a second gasbag in the at least two gasbags, and a non-gasbag watchband.

The MCU is further configured to: control the air pump to blow air at a preset rate; obtain a change rate of the differential pressure within second preset duration based on the differential pressure at the air nozzle collected by the differential pressure sensor; detect, based on the level output through the signal output pin and the change rate of the differential pressure within the second preset duration, whether the gasbag is mounted on the wearable device; and detect, based on the pin that outputs, through the signal output pin, the level greater than the second threshold and the change rate of the differential pressure within the second preset duration, an identifier of the gasbag mounted on the wearable device.

In a possible implementation, the differential pressure sensor includes a first pressure sensor and a second pressure sensor, the first pressure sensor is disposed at the air nozzle, the first pressure sensor is configured to detect a first pressure at the air nozzle, the second pressure sensor is configured to detect a second pressure, the second pressure is an environmental pressure, and the differential pressure at the air nozzle is a difference between the first pressure and the second pressure.

In a possible implementation, the positive pin, the negative pin, and the air nozzle are disposed on an outer surface of the wearable device.

It should be understood that the wearable device provided in this embodiment of this application may perform actions of the wearable device in the foregoing method embodiments. Implementation principles and technical effects thereof are similar. Details are not described herein again.

In an embodiment, an embodiment of this application further provides a wearable device. The wearable device may include a processor and a memory. The memory may include a high speed random-access memory (random-access memory, RAM), and may further include a non-volatile memory (non-volatile memory, NVM), for example, at least one disk memory. The memory may store various instructions, to complete various processing functions and implement the method steps of this application. It should be understood that, in an embodiment, the processor may not be limited to an MCU or a CPU. As shown in FIG. 2, in an embodiment, the wearable device may further include a Hall effect sensor and an air pump. As shown in FIG. 7, in an embodiment, the wearable device may further include a Hall effect sensor, an air pump, and a differential pressure sensor.

Optionally, the wearable device in this application may further include a power supply, a communication bus, and a communication port. The communications port is configured to implement connection and communication between the wearable device and another peripheral. In this embodiment of this application, the memory is configured to store computer-executable program code. The program code includes instructions. When the processor executes the instructions, the instructions enable the processor of the wearable device to perform the actions in the foregoing method embodiments. Implementation principles and technical effects thereof are similar. Details are not described herein again.

It should be noted that the modules or components in the foregoing embodiments may be one or more integrated circuits configured to implement the foregoing method, for example, one or more application-specific integrated circuits (application-specific integrated circuit, ASIC), one or more microprocessors (digital signal processor, DSP), or one or more field programmable gate arrays (field programmable gate array, FPGA). For another example, when one of the foregoing modules is implemented in a form of scheduling program code by a processing element, the processing element may be a general-purpose processor, for example, a central processing unit (central processing unit, CPU) or another processor that can invoke the program code, for example, a controller. For another example, the modules may be integrated together, and implemented in a form of a system-on-a-chip (system-on-a-chip, SOC).

All or some of the foregoing embodiments may be implemented by using software, hardware, firmware, or any combination thereof. When software is used to implement embodiments, all or a part of embodiments may be implemented in a form of a computer program product. The computer program product includes one or more computer instructions. When the computer program instructions are loaded and executed on a computer, all or some of the procedures or functions according to embodiments of this application are generated. The computer may be a general-purpose computer, a dedicated computer, a computer network, or another programmable apparatus. The computer instructions may be stored in a computer-readable storage medium or may be transmitted from a computer-readable storage medium to another computer-readable storage medium. For example, the computer instructions may be transmitted from a website, computer, server, or data center to another website, computer, server, or data center in a wired (for example, a coaxial cable, an optical fiber, or a digital subscriber line (DSL)) or wireless (for example, infrared, radio, or microwave) manner. The computer-readable storage medium may be any usable medium accessible by the computer, or a data storage device, for example, a server or a data center, integrating one or more usable media. The usable medium may be a magnetic medium (for example, a floppy disk, a hard disk drive, or a magnetic tape), an optical medium (for example, a DVD), a semiconductor medium (for example, a solid state disk (Solid State Disk, SSD)), or the like.

The term “a plurality of” in this specification means two or more than two. The term “and/or” in this specification describes only an association relationship for describing associated objects and represents that three relationships may exist. For example, A and/or B may represent the following three cases: Only A exists, both A and B exist, and only B exists. In addition, the character “/” in this specification usually indicates an “or” relationship between the associated objects. In the formula, the character “/” indicates a “division” relationship between the associated objects. In addition, it should be understood that in description of this application, terms such as “first” and “second” are merely used for distinguishing and description, but should not be understood as indicating or implying relative importance, or should not be understood as indicating or implying a sequence.

It may be understood that various numbers in embodiments of this application are merely used for differentiation for ease of description, and are not used to limit the scope of embodiments of this application.

It should be understood that sequence numbers of the foregoing processes do not mean execution sequences in embodiments of this application. The execution sequences of the processes should be determined based on functions and internal logic of the processes, and should not be construed as any limitation on the implementation processes of embodiments of this application.

1.-26. (canceled)

27. A method for a wearable device, the method comprising:
when a gasbag is mounted on the wearable device, detecting, based on a wrist circumference of a user, whether the gasbag mounted on the wearable device matches the wrist circumference of the user; and
in response to detecting that the gasbag mounted on the wearable device does not match the wrist circumference of the user, outputting first prompt information, wherein the first prompt information indicates to mount a gasbag that matches the wrist circumference of the user.

28. The method according to claim 27, further comprising:
detecting whether the gasbag is mounted on the wearable device; and
wherein detecting, based on the wrist circumference of the user, whether the gasbag mounted on the wearable device matches the wrist circumference of the user comprises:
in response to detecting that the gasbag is mounted on the wearable device, detecting an identifier of the gasbag mounted on the wearable device; and
detecting, based on a mapping relationship between one or more wrist circumferences and one or more identifiers of gasbags, whether the identifier of the gasbag mounted on the wearable device is an identifier of a gasbag mapped to the wrist circumference of the user.

29. The method according to claim 28, wherein detecting, based on the mapping relationship between the one or more wrist circumferences and the one or more identifiers of gasbags, whether the identifier of the gasbag mounted on the wearable device is the identifier of a gasbag mapped to the wrist circumference of the user comprises:
when the identifier of the gasbag mounted on the wearable device is not the identifier of the gasbag mapped to the wrist circumference of the user, determining that the gasbag mounted on the wearable device does not match the wrist circumference of the user; or
when the identifier of the gasbag mounted on the wearable device is the identifier of the gasbag mapped to the wrist circumference of the user, determining that the gasbag mounted on the wearable device matches the wrist circumference of the user.

30. The method according to claim 28, wherein the wearable device comprises a Hall effect sensor, and the Hall effect sensor comprises a positive pin, a negative pin, and a signal output pin, and the method comprises:
in response to sensing a positive magnetic pole, outputting, by the positive pin, through the signal output pin, a level greater than a second threshold;
in response to sensing a negative magnetic pole, outputting, by the negative pin, through the signal output pin, the level greater than the second threshold;
in response to the positive pin not sensing the positive magnetic pole, outputting, by the positive pin, through the signal output pin, a level less than a first threshold, wherein the second threshold is greater than the first threshold; and
in response to the negative pin not sensing the negative magnetic pole, outputting, by the negative pin, through the signal output pin, the level less than the first threshold.

31. The method according to claim 30, wherein the wearable device comprises at least two gasbags, magnetic poles are disposed on the gasbags of the at least two gasbags, and a different magnetic pole is disposed on each gasbag;
wherein detecting whether the gasbag is mounted on the wearable device comprises:
detecting, based on the level output through the signal output pin, whether the gasbag is mounted on the wearable device; and
wherein detecting the identifier of the gasbag mounted on the wearable device comprises:
detecting, based on which pin outputs, through the signal output pin, the level greater than the second threshold, the identifier of the gasbag mounted on the wearable device.

32. The method according to claim 31, wherein the at least two gasbags comprise a first gasbag and a second gasbag, the negative magnetic pole is disposed on the first gasbag, the positive magnetic pole is disposed on the second gasbag, and detecting, based on the level output through the signal output pin, whether the gasbag is mounted on the wearable device comprises:
in response to the level output through the signal output pin being less than the first threshold, determining that no gasbag is mounted on the wearable device; or
in response to the level output through the signal output pin being greater than the second threshold, determining that the gasbag is mounted on the wearable device.

33. The method according to claim 32, wherein detecting, based on which pin outputs, through the signal output pin, the level greater than the second threshold, the identifier of the gasbag mounted on the wearable device comprises:
in response to the positive pin outputting, through the signal output pin, the level greater than the second threshold, determining that the gasbag mounted on the wearable device is the second gasbag; or
in response to the negative pin outputting, through the signal output pin, the level greater than the second threshold, determining that the gasbag mounted on the wearable device is the first gasbag.

34. The method according to claim 28, wherein the wearable device comprises a differential pressure sensor and a gasbag watchband, the differential pressure sensor is configured to detect a differential pressure at an air nozzle of an air pump in the wearable device, the gasbag is mounted on the gasbag watchband, and when the gasbag watchband on which the gasbag is mounted is mounted on the wearable device, an air intake on the gasbag communicates with the air nozzle;
wherein detecting whether the gasbag is mounted on the wearable device comprises:
controlling the air pump to blow air at a preset rate;
obtaining a change rate of the differential pressure within a second preset duration based on the differential pressure at the air nozzle collected by the differential pressure sensor; and
detecting, based on the change rate of the differential pressure within the second preset duration, whether the gasbag is mounted on the wearable device; and
wherein detecting the identifier of the gasbag mounted on the wearable device comprises:
detecting, based on the change rate of the differential pressure within the second preset duration, the identifier of the gasbag mounted on the wearable device.

35. The method according to claim 34, wherein the wearable device further comprises a non-gasbag watchband, and when the non-gasbag watchband is mounted on the wearable device, the non-gasbag watchband blocks the air nozzle of the air pump; and
wherein detecting, based on the change rate of the differential pressure within the second preset duration, whether the gasbag is mounted on the wearable device comprises:
when the change rate of the differential pressure within the second preset duration is o, or the change rate of the differential pressure within the second preset duration is greater than or equal to a first change rate threshold, determining that no gasbag is mounted on the wearable device; or
when the change rate of the differential pressure within the second preset duration is less than the first change rate threshold, determining that the gasbag is mounted on the wearable device.

36. The method according to claim 35, wherein detecting, based on the change rate of the differential pressure within the second preset duration, the identifier of the gasbag mounted on the wearable device comprises:
in response to information that the change rate of the differential pressure within the second preset duration is less than the first change rate threshold, detecting, based on the change rate of the differential pressure within the second preset duration and a mapping relationship between one or more change rates of a differential pressure and one or more identifiers of gasbags, the identifier of the gasbag mounted on the wearable device.

37. The method according to claim 30, wherein the wearable device comprises:
a differential pressure sensor, a gasbag watchband, and at least two gasbags, the differential pressure sensor is configured to detect a differential pressure at an air nozzle of an air pump in the wearable device, the gasbag is mounted on the gasbag watchband, when the gasbag watchband on which the gasbag is mounted is mounted on the wearable device, an air intake on the gasbag communicates with the air nozzle, and magnetic poles disposed on at least two of the following are the same: a first gasbag in the at least two gasbags, a second gasbag in the at least two gasbags, or a non-gasbag watchband;
wherein detecting whether the gasbag is mounted on the wearable device comprises:
controlling the air pump to blow air at a preset rate;
obtaining a change rate of the differential pressure within second preset duration based on the differential pressure at the air nozzle collected by the differential pressure sensor; and
detecting, based on the level output through the signal output pin and the change rate of the differential pressure within the second preset duration, whether the gasbag is mounted on the wearable device; and
wherein detecting the identifier of the gasbag mounted on the wearable device comprises:
detecting, based on which pin outputs, through the signal output pin, the level greater than the second threshold and the change rate of the differential pressure within the second preset duration, the identifier of the gasbag mounted on the wearable device.

38. The method according to claim 37, wherein the negative magnetic pole is disposed on the first gasbag, the positive magnetic pole is disposed on the second gasbag, and the positive magnetic pole is disposed on the non-gasbag watchband; and
wherein detecting, based on the level output through the signal output pin and the change rate of the differential pressure within the second preset duration, whether the gasbag is mounted on the wearable device comprises:
in response to the level output through the signal output pin being less than the first threshold, determining that no gasbag is mounted on the wearable device;
in response to the level output through the signal output pin being greater than the second threshold, and the negative pin outputting, through the signal output pin, the level greater than the second threshold, determining that the gasbag mounted on the wearable device is the first gasbag; or
in response to the level output through the signal output pin being greater than the second threshold, and the positive pin outputting, through the signal output pin, the level greater than the second threshold, detecting, based on the change rate of the differential pressure within the second preset duration, that the second gasbag or the non-gasbag watchband is mounted on the wearable device.

39. The method according to claim 38, wherein when the non-gasbag watchband is mounted on the wearable device, the non-gasbag watchband blocks the air nozzle of the air pump; and
wherein detecting, based on the change rate of the differential pressure within the second preset duration, that the second gasbag or the non-gasbag watchband is mounted on the wearable device comprises:
in response to the change rate of the differential pressure within the second preset duration being greater than or equal to a first change rate threshold, determining that the non-gasbag watchband is mounted on the wearable device; or
in response to the change rate of the differential pressure within the second preset duration being less than the first change rate threshold, determining that the gasbag mounted on the wearable device is the second gasbag.

40. The method according to claim 34, wherein the differential pressure sensor comprises a first pressure sensor and a second pressure sensor, the first pressure sensor is configured to detect a first pressure at the air nozzle, the second pressure sensor is configured to detect a second pressure, the second pressure is an environmental pressure, and the differential pressure at the air nozzle is a difference between the first pressure and the second pressure; and
wherein obtaining the change rate of the differential pressure within the second preset duration based on the differential pressure at the air nozzle collected by the differential pressure sensor comprises:
obtaining one or more first pressures collected by the first pressure sensor based on an interval during a first preset duration;
obtaining one or more second pressures collected by the second pressure sensor, each second pressure being collected based on a time interval having a first preset duration, and one second pressure being collected in each respective time interval;
obtaining a first pressure average value based on first pressures collected within the second preset duration;
obtaining a second pressure average value based on second pressures collected within the second preset duration; and
obtaining the change rate of the differential pressure within the second preset duration based on the first pressure average value and the second pressure average value.

41. A wearable device, comprising:
a Hall effect sensor, wherein the Hall effect sensor comprises a positive pin, a negative pin, and a signal output pin;
wherein the Hall effect sensor is configured to:
in response to sensing a positive magnetic pole, outputting, by the positive pin through the signal output pin, a level greater than a second threshold;
in response to sensing a negative magnetic pole, outputting, by the negative pin through the signal output pin, the level greater than the second threshold;
in response to the positive pin not sensing the positive magnetic pole, outputting, by the positive pin through the signal output pin, a level less than a first threshold, wherein the second threshold is greater than the first threshold; and
in response to the negative pin not sensing the negative magnetic pole, outputting, by the negative pin through the signal output pin, the level less than the first threshold.

42. The wearable device according to claim 41, wherein the wearable device comprises at least two gasbags and a microprocessor, and the microprocessor is connected to the Hall effect sensor; and
the microprocessor is configured to detect the level output through the signal output pin.

43. The wearable device according to claim 42, wherein magnetic poles are disposed on the gasbags of the at least two gasbags, and a different magnetic pole is disposed on each gasbag; and
the microprocessor is further configured to:
detect, based on the level output through the signal output pin, whether the gasbag is mounted on the wearable device; and
detect, based on which pin outputs, through the signal output pin, the level greater than the second threshold, an identifier of the gasbag mounted on the wearable device.

44. The wearable device according to claim 42, wherein the wearable device further comprises a differential pressure sensor and a gasbag watchband, the differential pressure sensor is configured to detect a differential pressure at an air nozzle of an air pump in the wearable device, the gasbag is mounted on the gasbag watchband, and when the gasbag watchband on which the gasbag is mounted is mounted on the wearable device, an air intake on the gasbag communicates with the air nozzle; and
the microprocessor is further configured to:
control the air pump to blow air at a preset rate;
obtain a change rate of the differential pressure within a second preset duration based on the differential pressure at the air nozzle collected by the differential pressure sensor;
detect, based on the change rate of the differential pressure within the second preset duration, whether the gasbag is mounted on the wearable device; and
detect, based on the change rate of the differential pressure within the second preset duration, an identifier of the gasbag mounted on the wearable device.

45. The wearable device according to claim 42, further comprising:
a differential pressure sensor, a gasbag watchband, and the at least two gasbags;
wherein the differential pressure sensor is configured to detect a differential pressure at an air nozzle of an air pump in the wearable device, the gasbag is mounted on the gasbag watchband, when the gasbag watchband on which the gasbag is mounted is mounted on the wearable device, an air intake on the gasbag communicates with the air nozzle, and magnetic poles disposed on at least two of the following are the same: a first gasbag in the at least two gasbags, a second gasbag in the at least two gasbags, or a non-gasbag watchband; and
the microprocessor is further configured to:
control the air pump to blow air at a preset rate;
obtain a change rate of the differential pressure within a second preset duration based on the differential pressure at the air nozzle collected by the differential pressure sensor;
detect, based on the level output through the signal output pin and the change rate of the differential pressure within the second preset duration, whether the gasbag is mounted on the wearable device; and
detect, based on which pin outputs, through the signal output pin, the level greater than the second threshold and the change rate of the differential pressure within the second preset duration, an identifier of the gasbag mounted on the wearable device.

46. The wearable device according to claim 44, wherein the differential pressure sensor comprises a first pressure sensor and a second pressure sensor, the first pressure sensor is disposed at the air nozzle, the first pressure sensor is configured to detect a first pressure at the air nozzle, the second pressure sensor is configured to detect a second pressure, the second pressure is an environmental pressure, and the differential pressure at the air nozzle is a difference between the first pressure and the second pressure.
